(19) United States
(12) Patent Application Publication
(10) Pub. No.: US 2023/0212326 A1
(43) Pub. Date: Jul. 6, 2023

(54) PECTIN EXTRACTION PROCESS

(71) Applicant: DUPONT NUTRITION BIOSCIENCES APS, Kongens Lyngby (DK)

(72) Inventors: Kenneth Boetkjaer, Kongens Lyngby (DK); Alex Andreassen, Kongens Lyngby (DK)

(21) Appl. No.: 17/997,932

(22) PCT Filed: May 12, 2021
(86) PCT No.: PCT/EP2021/062665

§ 371 Date: Nov. 4, 2022

(30) Foreign Application Priority Data
May 14, 2020 (EP) 20174682.3

(51) Int. Cl. C08B 37/00; C12P 19/04; C12P 19/14; A23L 29/231

(52) U.S. Cl. CPC C08B 37/0048; C12P 19/04; C12P 19/14; A23L 29/231

(57) ABSTRACT

The invention relates to a process for reducing the reactivity of pectin extracted from a raw material containing a high proportion of calcium-sensitive pectin, the process comprising subjecting the extracted pectin to enzymatic depolymerization.

### FIELD OF THE INVENTION

This invention relates to a process for extracting pectin from a pectin-containing raw material. The invention further relates to the use of the pectin obtained in this process.

### BACKGROUND OF THE INVENTION

Pectin is a commonly used additive in the food industry. It is useful, for example, as a stabilizing agent, thickener and gelling agent in, for example, jams, jellies and other fruit-based products as well as in acidified milk-based products such as yogurts. Pectin has also found other uses in the food industry, for example as a fat replacer.

Pectin is a structural polysaccharide typically found in the form of a water insoluble parent pectic substance—protopectin—in the primary cell wall and the middle lamella of green land plants such as fruit and vegetables. Major sources of commercial pectin products are citrus peel and apple pomace in which protopectin represents 10-40% by weight of the dry matter.

Pectin is the generic designation for water-soluble compounds which result from restricted hydrolysis of protopectin. The exact nature of protopectin is not completely understood. It is, however, generally recognized that protopectin is a complex structure in which pectin is attached to other cell wall components such as cellulose, cell wall protein and hemicellulose by covalent bonds, hydrogen bonds and/or ionic interactions.

Pectin molecules have a molecular weight of up to more than 200,000 Da and a degree of polymerization of up to more than 1000 units. A proportion of the carboxylic acid groups of the galacturonic acid units are methyl-esterified. In plants the residual carboxyl groups are partly or completely neutralized with cations of calcium, potassium and magnesium which inherently are contained in the plant tissues.

The source of pectin will govern to some extent whether or not other ester groups are present in the pectin structure. In this respect, it is known that some pectins comprise acetyl groups. Here, typically, the hydroxyl groups on C₂ or C₃ may be acetylated. By way of example, the sugar beet pectin is to some extent acetylated at O-2 and/or O-3 of the galacturonic acid residues.

The structure of pectin, in particular the degree of esterification, determines its physical and/or chemical properties. For example, pectin gelation depends on the chemical nature of pectin, especially the degree of esterification and degree of polymerization. In addition, however, pectin gelation also depends on the pectin concentration and environmental conditions like soluble solids content, the pH and calcium ion concentration.

Furthermore, according to the Food Chemicals Codex, in order to be used in the food industry, the pectin should have a minimum 65% of galacturonic acid on dry and ash-free basis. Thus, the pectin obtained by the process of the present invention is suitable for food applications.

Pectin is commonly extracted as a bulk-extracted pectin fraction showing molecular variability and following a varied interaction between calcium ions and separate pectin molecules having varying affinity towards calcium ions and other charged particles. The bulk-extracted pectin products comprise molecules that represent a broad distribution of esterification degree and molecules of different methyl esterification patterns. This affects the important quality parameters for pectin such as breaking strength of gels, setting temperature profile, interactions with protein or cations and solubility of pectin in food manufacturing applications.

In the document WO 2015/091629, it is described a process for pectin extraction in two steps, in which the first step of extraction provides calcium-tolerant pectin and the second step of extraction provides calcium-sensitive pectin. In some applications, the high calcium-sensitivity of the pectin from second extraction creates undesirable effects such as pre-gelation and inhomogeneous mixing during food production. The wide distribution of pectin calcium-sensitivity that is present in the product obtained by the process of the mentioned prior art documents creates challenges in application. The calcium-tolerant pectin fraction will be suitable for some applications, whereas the calcium-sensitive fraction is suitable for different applications.

So, the present invention is an improvement of the process described in the state of the art in which the second extract or the combined first and second extracts are submitted to a third step of enzymatic treatment. The exact sequence of extractions and enzymatic treatment is important since it affects the final functionality. Thus, the molecular structure of pectin is fine-tuned by the process described herein. If the first fraction is also treated enzymatically, this negatively affect functionality in certain food applications. In other applications, a treatment of the combined first and second fraction will in general reduce the distribution of calcium-sensitivity and improve final functionality.

Some enzymatic treatments are known in the art, as for examples in the documents “Rolin, Gums and Stabilisers for the Food Industry, 1994, 413-422” and “Westerng et al. Carbohydrate Polymers 72 (2008) 32-42”, describing the use of polygalacturonase for treatment of high ester pectin to reduce calcium-sensitivity. However, the selective enzymatic treatment of second extract, as described herein, is imperative for some pectin applications and if not followed specifically the enzymatic treatment will destroy the whole functionality. This is not obvious since it is necessary to adapt the treatment to adjust the reactivity/functionality of the final product. In addition, the process described herein also include the combined treatment of pectin extracts with polygalacturonase to lower calcium-reactivity and pectin methyl esterase to reduce the degree of esterification. The process therefore is not limited to a reduction of calcium-sensitivity of high-ester pectin, but also includes the reduction of calcium-reactivity of low-ester pectin products when a first and a second extraction step is utilized.

However, many pectin applications require homogeneous pectin fractions but this results in a relatively low pectin yield and high production costs.

A demand is present in the industry for ways to obtain a high extraction yield along with controlled and consistent pectin functionality both from an environmental and a sustainable as well as an economic point of view due to the efficient use of the pectin containing resources.

### SUMMARY OF THE INVENTION

It is the object of the invention to provide a process for extraction and/or isolation of pectin, where the process results in an increased calcium tolerance and lower protein reactivity of the extracted pectin. It is furthermore an object of the invention to provide a more homogenous pectin fraction with shorter galacturonic acid block structures in the polymer chain.

Accordingly, the present invention relates to a process for reducing the reactivity of pectin extracted from a pectin-containing raw material containing a high proportion of calcium-sensitive pectin, the process comprising the steps of
(a) subjecting a pectin-containing raw material containing a high proportion of calcium-sensitive pectin to a first extraction procedure using a first aqueous solution of a pH in the range of 1.0-2.5, followed by separation of a first pectin-containing residue from the first aqueous solution, resulting in a first extract comprising both calcium-sensitive and calcium-tolerant pectin,
(b) subjecting, optionally after a washing step, the first pectin-containing residue to a second extraction procedure using a second aqueous solution of a pH in the range of 3.0-6.0, said second aqueous solution optionally comprising a chelating agent, followed by separation of a second pectin-containing residue from the second aqueous solution, resulting in a second extract comprising calcium-sensitive pectin, and
(c) subjecting the second extract of step (a) or the combined first and second extracts of step (a) and (b) to treatment with an enzyme capable of depolymerizing pectin before or during purification of the extracts resulting in a pectin fraction comprising calcium-tolerant pectin with a lower molecular weight than the pectin present in the pectin-containing raw material.

It has been found that the enzymatic depolymerization results in extraction of a high yield of calcium-tolerant pectin from a pectin-containing raw material containing a high proportion of calcium sensitive pectin.

The present process is particularly favourable because it combines a higher extraction yield with properties of the extracted pectin that are similar to those of conventional acid extracted pectin (typically extracted in a lower yield) so that it can be used for many applications where pectin from conventional extraction processes is normally used.

### BRIEF DESCRIPTION OF THE DRAWINGS

FIG. 1.: Rapid Viscosity Analyser (RVA) on LC pectins. Viscosity (cP) was measured for the samples described in table 4 at a range of temperatures between 55-85° C. using RVA. #1 4963430719, #2 3834-126-8, #3 3834-126-7, #4 3834-126-6.

### DETAILED DESCRIPTION OF THE INVENTION

The term “pectin” is to be understood as a water-soluble form of pectic substance obtained by extraction of pectin from a plant material. Pectin has a structure comprising blocks of linear galacturonan chains (polymer of α-(1-4)-linked-D-galacturonic acid) which are interrupted with rhamno-galacturonan backbones (polymers of the repeating disaccharide α-(1-4)-D-galacturonic acid-α-(1-2)-L-rhamnose), which often have side chains of polymeric arabinogalactans glycosidic linked to the O-3 or O-4 positions of L-rhamnose. The galacturonan sequences can have D-xylose and D-apiose glycosidic linked to their O-2 or O-3 positions, which also can be substituted with ester-linked acetyl groups. The long chains of α-(1-4)-linked D-galacturonic acid residues are commonly referred to as “smooth regions”, whereas the highly branched rhamnogalacturonan regions are commonly referred to as the “hairy regions”.

The “degree of esterification” (DE) means the extent to which free carboxylic acid groups contained in the galacturonic acid units of pectin have been methyl esterified. The resultant pectin is referred to as “high ester pectin” (“HE pectin” for short) if more than 50% of the carboxyl groups are esterified. The resultant pectin is referred to as a “low ester pectin” (“LE pectin” for short) if less than 50% of the carboxyl groups are esterified. If the pectin does not contain any—or only a few—esterified groups it is usually referred to as pectic acid.

Pectins with a block structure of linear galacturonan chains exhibit increased calcium reactivity where galacturonic acid residues of one pectin molecule cross-link with galacturonic acid residues of another pectin molecule via calcium ions to form dense network structures. “Calcium reactivity” (also termed “calcium sensitivity”) of pectin therefore results in increase of viscosity of a pectin solution in presence of calcium ions

The content of calcium sensitive pectin in a pectin product can be described as the “calcium sensitive pectin ratio” (CSP-ratio or CSPR), which is the fraction of calcium-sensitive pectin in a pectin product. Pure calcium-sensitive pectin therefore theoretically has CSPR=1.0, a pectin which has 50% calcium-sensitive pectin has CSPR=0.5 and a pure calcium-tolerant pectin has CSPR=0. In the present context, “calcium-tolerant pectin” (also termed “non-calcium sensitive pectin” in the literature) is meant to include pectin that exhibits increased calcium tolerance and is significantly less reactive than calcium-sensitive pectin present in the pectin-containing extracts of step (a) and (b) of the present process prior to enzyme treatment, whereby the galacturonan blocks are cleaved resulting in shorter blocks making the pectin less calcium reactive. By way of example, the CSPR has been reduced from about 0.85 to about 0.65 in pectin prepared by the process described in Example 1 below.

The term “enzyme capable of depolymerizing pectin” is understood to mean any enzyme that acts on blocks of galacturonan chains of pectin, thereby reducing the degree of polymerization and the molecular weight of pectin.

The term “exo-polygalacturonase” is understood to mean an enzyme which depolymerizes pectin by hydrolysis of non-methylesterified homogalacturonan in which the enzyme act on the end of the polymer and usually on the non-reducing end.

The term “endo-polygalacturonase” is understood to mean an enzyme which depolymerizes pectin by hydrolysis of non-methylesterified homogalacturonan in which the enzyme acts in the middle of the oligo/polysaccharide chain

The term “pectate lyase” is understood to mean an enzyme which depolymerizes pectin by a β-elimination reaction of non-methylesterified homogalacturonan.

The pectin-containing raw material can preferably be obtained from citrus fruits, apples, sugar beets, sunflower heads, vegetables or waste products from plants such as apples, sugar beets, carrots, onions, peaches, grape berries, mangos, guavas, squashes, pumpkins, tomatoes, apricots, bananas, beans, potatoes, sunflower or citrus fruits. Examples of citrus fruits are limes, lemons, grapefruits, mandarins, tangerines, pomelos, and oranges.

In order to obtain the pectin-containing raw material, the fruits or vegetables are treated depending on the type of fruit and vegetable in a manner commonly known by persons skilled in the art. This may comprise disintegrating or pressing the material to separate juice and oil, followed by several washes with water to remove soluble solids like sugars and residues of oil and fat. The washed material can be pressed and used directly for pectin extraction or dried to more than 85% dry matter to be safe for transport or storage.

In one embodiment, the pectin-containing raw material includes dried peel from citrus fruits, having a dry matter content of about 85% by weight or more, preferably in the form of pieces of at the most 2 cm in length, which are obtained by processing pressed peel from the juice industry after extraction of the citrus juice and the essential oils. These materials all have a high content of pectic substances in the form of water-insoluble protopectin. Citrus peel having a content of extractable pectin in the range of 20-35% by weight on a dry matter basis is particularly interesting.

The pectin-containing raw material may favourably be subjected to a pre-treatment in order to change the particle size distribution of the raw material by milling the dry peel or by cutting down the raw material in an aqueous peel suspension.

Drying the material can be carried out by means of any conventional drying equipment such as a drying oven, a belt dryer, a drum dryer or a fluid bed dryer for a period of time sufficient to obtain a dry matter content in the material of at least 80% by weight. The dry matter content in the material is preferably at least 85% by weight, more preferably at least 90% by weight. The drying may be carried out at a temperature ranging from ambient temperature to above 100° C. for a period of at most 36 hours. For instance, the drying may be carried out at a temperature in the range of from 40° C. to 100° C., for a period of at most 36 hours. The drying may be carried out at pressures below atmospheric pressure, whereby a relatively lower drying temperature or a comparatively shorter drying period can be used resulting in a more gentle treatment.

In step (a) of the present process, an acid extraction is performed with a first aqueous solution with a pH in the range of 1.0-2.5 resulting in an extraction of both calcium tolerant pectin and calcium sensitive pectin from the raw material into the aqueous solution. The pH of said first aqueous solution may suitably be from 1.5 to 2.5. In a currently preferred embodiment, the pH of said first aqueous solution is from 2 to 2.5.

In order to increase the yield of extracted pectin, the extraction temperature and time of extraction can be adjusted. Thus, step (a) of the process may be carried out at a temperature between 15-100° C., such as a temperature between 65-75° C.

The acid extraction procedure of step (a) may suitably be carried out for a period of time from 0.5 to 10 hours, in particular a period of time from 1 to 8 hours.

The amount of the first aqueous solution to be used in step (a) depends for example on the origin and the condition of the pectin-containing material to be extracted and the content of extractable pectin in the material.

Both organic or inorganic acids may be used in step (a). Useful acids include strong or medium strong inorganic acids as exemplified by but not limited to hydrochloric acid, sulphuric acid, sulphurous acid, nitric acid or phosphoric acid. Organic acids include but are not limited to formic acid, acetic acid, propionic acid, citric acid, malonic acid, succinic acid, tartaric acid, oxalic acid, glyoxalic acid, lactic acid, glycerolic acid, maleic acid, fumaric acid and benzoic acid. It is to be understood that mixtures of acids may be used.

It has been found that a second extraction of the pectin-containing residue from step (a) minus the pectin fraction extracted by the acidic solution results in the release of a considerable amount of pectin in addition to the acid-extracted pectin fraction. The pectin extracted in step (b) of the present process is significantly different from the first extract with respect to chemical characteristics as well as functional pectin properties. Extraction step (b) appears to remove pectic material and create access to extraction of more calcium-sensitive pectin fractions which cannot be extracted by exclusively using a single acid extraction.

In order to obtain a high yield of calcium-sensitive pectin it is important that the pH of the aqueous solution used in step (b) is higher than the pH of the aqueous solution used in step (a). In one embodiment, the pH of the aqueous solution used in step (b) is from 3.0 to 6.0, in particular from 3.5 to 4.5, i.e. the second aqueous solution is a weak acidic solution. To increase the pH in the second solution, one or more bases may be added to the solution from step (a). The base may be a basic salt with mono- or divalent cations. Examples of such are carbonates, hydrogen carbonates as well as hydroxides of lithium, sodium, potassium, ammonium, calcium and magnesium.

In an embodiment, the second aqueous solution may comprise one or more of salts of partially neutralized inorganic acids like polyphosphate, borate, phosphate, pyrophosphate, phosphonates, hydrogen carbonate, hydrogen sulphate, zeolites, cationic organic resins, salts of partially neutralized organic acids or polycarboxylates such as tartrate, citrate, oxalate, gluconate, EDTA (ethylenediamine tetra acetic acid), DTPA (diethylenetriamine penta acetic acid), NTA (nitrilo triacetate), imidazole and derivates.

In an embodiment, the second aqueous solution may comprise one or more chelating agents and thus has a chelating function, i.e. it is able to form complexes with di- or multivalent cations.

Hereby, the di- or multivalent cations are bound by the chelating agents used for extraction step (b) and the calcium-sensitive pectin remains soluble. Thus, a higher fraction of the calcium-sensitive pectin is extracted resulting in a higher yield of calcium-sensitive pectin. It has been found that extraction step (b) appears to remove pectic material and create access to extraction of more calcium-sensitive pectin fractions by chelating agents.

In an embodiment, the chelating agent is selected from one or more of the following: polyphosphates, citric acid or its salts, oxalic acid or its salts, phosphoric acid or its salts, salts of partly neutralized di- or multivalent organic acids like EDTA, CDTA, DTPA, NTA, imidazole, carbonic acid or cationic ion exchange resins.

The second extraction procedure may suitably be carried out for a period of time from 0.5 to 5 hours.

The temperature of said second extraction procedure may suitably be between 50° C. and 80° C., preferably between 65° C. and 75° C.

The amount of pectin-containing material and aqueous solution in each extraction step, which is carried out while gently stirring the suspension of pectin-containing raw material, is selected so that the suspension has a dry matter content which is in the range of 1% to 20% by weight e.g. in the range of from 2% to 15% by weight such as in the range of from 2% to 6% by weight.

In an embodiment, at least one washing step may be included in the process between step (a) and step (b) or between step (b) and step (c). The washing step removes remains of the first aqueous (acidic) solution and the extracted (or solubilized) calcium-tolerant pectin and extracts some additional pectin from the remaining pectin-containing raw material. The washing step may be carried out using, for instance, continuous wash on a stationary filter, as a counter-current wash or as a wash by a percolation process. The washing step may be repeated, if necessary. The washing solution containing the extracted pectin may subsequently be combined with the first and second extracts.

After the second extraction in step (b), the second extract (and optionally the extracted pectin from the washing step) may either be subjected separately to purification and enzyme treatment in step (c), or the first and second extracts (and optionally the extracted pectin from the washing step) may be combined and then subjected to purification and enzyme treatment in step (c). The purification procedure typically involves centrifugation, clarification, filtration, ion exchange, concentration, precipitation, washing, pressing, drying and milling. All of these methods can be performed as known to the person skilled in the art.

Enzyme treatment of the second extract or of the combined first or second extracts according to the present process may favourably take place either before or after centrifugation in step (c) or after concentration of the second extract or of the combined first and second extracts in step (c).

In an embodiment of step (c), enzyme treatment is carried out after centrifugation of the second extract or combined first and second extracts. In this embodiment, enzyme treatment is preferably carried out before clarification and filtration of the second extract or combined first and second extracts. It has been found that when the enzyme treatment is applied at this stage during purification of the second extract or combined first and second extracts, the viscosity of the second extract or combined first and second extracts is reduced thereby facilitating purification downstream of process step (c). In the absence of enzyme treatment, the capacity of subsequent unit operations will be limited by increased viscosity. In an alternative embodiment, enzyme treatment may be carried out after filtration and before ion exchange.

In yet another embodiment of step (c), concentration of the second extract or combined first and second extracts conveniently takes place before addition of the enzyme. Concentration may for example be carried out by membrane filtration (such as ultrafiltration) or by evaporation under reduced pressure.

In step (c) the enzyme treatment results in a pectin fraction which is more calcium tolerant than the pectin in the starting raw material. The resulting calcium-tolerant pectin has a somewhat lower molecular weight than the pectin present in the raw material, such as a molecular weight about 5-100 kDa lower than that of the pectin in the raw material. The molecular weight reduction is controlled so that it does not negatively affect the functional properties of the pectin such as viscosity and gelling capacity. A higher processability can be obtained from the reduction in molecular weight. As appears from Example 1, the enzyme treatment does not reduce the % DE while the molecular weight is reduced compared to the pectin present in the raw material.

The pH during the enzyme treatment will depend upon the enzyme used in the process. If the pH used during the enzyme treatment is close to the pH optimum of the enzyme, the efficiency of the enzyme will be higher than when the pH is not at the pH optimum for the enzyme. This results for example in a lower amount of enzyme or a shorter time of enzyme treatment can be used to obtain the same amount of pectin.

In an embodiment, the enzyme treatment is carried out using a single purified enzyme. Hereby, the pectin modification can be better controlled and less unspecific breakdown of the pectin is observed, such as depolymerization of pectin in methyl esterified regions, breakdown of hairy regions or de-esterification. Hence, a homogenous pectin fraction can be obtained.

Examples of enzymes to be used in step (c) of the present process are polygalacturonase (exo-polygalacturonase or endo-polygalacturonase) or pectate lyase.

In a further embodiment, the enzyme treatment is carried out using a mixture of two or more enzymes. Hereby, the advantages of different enzymatic processes can be utilized to provide pectins with different functionalities can be obtained. In this embodiment, step (c) further comprises subjecting the second extract or combined first and second extracts to treatment with one or more enzymes capable of de-esterifying pectin resulting in a pectin extract comprising less calcium reactive pectin with both a reduced molecular weight and reduced degree of esterification compared to the pectin present in the pectin-containing raw material. Examples of enzymes that are useful for further modification of pectin functionality include pectin methyl esterase, pectin acetyl esterase, rhamnogalacturonase, galactanase, arabinase and rhamnogalacturonan hydrolase.

As a preferred embodiment of the invention, the pectin can be specifically modified during the process to increase the de-esterification by means of an esterase such as pectin methyl esterase and hereby obtain an LE pectin. Pectin methyl esterase can be added together with cellulase which increases the release of pectin from the second pectin-containing residue. Thus, an increased yield of calcium-tolerant LE pectin can be obtained by mixing for instance polygalaturonase and pectin methyl esterase.

The resulting LE pectin may, if desired, be further modified by amidation using, e.g., NH3 resulting in amidated LE pectin which typically has a higher gel strength than non-amidated LE pectin.

It may generally be preferred that the pH of said enzyme-containing solution is higher than the pH of said first aqueous solution. Thus, the pH during the enzyme treatment is higher than the pH during the acid extraction. Thus, the pH of said enzyme-containing solution is from 2.0 to 5.5, such as from 2.5 to 4.5.

In one aspect, the enzyme treatment is carried out for a period of time from 0.5 to 20 hours.

The enzyme treatment of step (c) may typically be carried out at a temperature between 40° C. and 80° C., preferably 45-60° C., such as 50-55° C.

The amount of depolymerizing enzyme added to the second extract or combined first and second extracts before or after centrifugation in step (c) may vary according to the type of enzyme used as well as the process conditions, in particular with respect to temperature and time, but is typically in the range of 0.001-2 units/L extract (the method of determining enzyme activity in units/mL is described below).

The amount of depolymerizing enzyme added after concentration of the second extract or combined first and second extracts in step (c) may vary according to the type of enzyme used as well as the process conditions, in particular temperature and time, but is typically in the range of 0.005-10 units/L concentrate.

The amount of de-esterification enzyme added to the second extract or combined first and second extracts before or after centrifugation in step (c) may vary according to the type of enzyme used as well as the process conditions, in particular with respect to temperature and time, but is typically in the range of 2-2000 units/L extract.

The amount of de-esterification enzyme added after concentration of the second extract or combined first and second extracts in step (c) may vary according to the type of enzyme used as well as the process conditions, in particular temperature and time, but is typically in the range of 10-10.000 units/L concentrate.

The precipitation of pectin can be performed with any solvent which is water-miscible and in which pectin is substantially insoluble, e.g. 2-propanol or another alcohol or a ketone. The precipitated pectin is separated from the liquid by any convenient method, such as for example by decantation, centrifugation or filtration, and the precipitate can then be pressed and washed on the filter to remove soluble salts and impurities. Finally, the pectin can be dried and optionally ground before use.

This invention furthermore relates to the use of calcium-tolerant pectin as obtained by the process described above where the pectin is used for stabilizing beverages, structuring of acidified dairy products, as a dietary fiber, a gelling agent for jams and jellies, a stabilizer in dairy desserts and/or a protein complexing agent. It has been found that pectin subjected to polygalacturonase and pectin methyl esterase treatment according to step (c) of the present process exhibits reduced gelation temperatures compared to pectin treated with pectin methyl esterase alone. Using a pectin prepared by the present process has consequently made it possible to avoid pre-gelation when making gelled food products such as jams and jellies.

The beverage may for instance be acidified milk or diluted fruit juices.

In one embodiment, the acidified milk products are selected from acidified milk products e.g. having a pH of 3.5 to 5, such as acidified milk, drinking yogurt and yogurt with fruit.

The acidified milk products comprise milk products obtained by acidification either through fermentation with live acid producing bacteria or by addition of fruit juices or food acids.

Furthermore, a use is described where the pectin is used as a stabilizer in dairy desserts.

The pectin can further be used as soluble fibre (optionally: as Ca-salt), carrier of nutrients (Fe, Ca, Zn), binder of radionuclides in food, precursor for conventional low ester pectin or amidated pectin, protein binder for protein recovery from process water in food plants and protein flocculant in waste water plants, encapsulation agent and dye binder.

### Methods

### Determination of Degree of Esterification (DE)

The degree of esterification was determined according to the procedures given in Food Chemical Codex, Third Edition, National Academic Press, Washington 1981, page 216.

Weigh 5 g of the pectin sample to the nearest 0.1 mg into a 250 ml beaker and add a mixture of 100 ml 60% aqueous 2-propanol and 5 ml conc. hydrochloric acid. Stir on a magnetic stirrer for 10 minutes. Filter through a dried and pre-weighed 30 ml coarse glass filter funnel with reduced vacuum. Wash with six 15 ml portions of HCl-60% 2-propanol mixture. Then wash with 60% aqueous 2-propanol (6-8 portions of 20 ml) until the filtrate is free from chloride (test with a solution of 1.7 g silver nitrate in 100 ml of distilled water). Finally wash with approx. 30 ml of 100% 2-propanol. Dry for 2½ hours in an oven at 105° C. Cool in a desiccator. Weigh.

Pipette 20.00 ml of 0.5 N sodium hydroxide using 20 ml volumetric pipette into a beaker and mix with 20.00 ml of 0.5 N hydrochloric acid, which has been transferred using a 20 ml volumetric pipette. Add two drops of a solution of phenolphthalein (1 g of phenolphthalein is dissolved in 100 ml of 96% ethanol) indicator and titrate with 0.1 N sodium hydroxide. Record the volume V₀.

Weigh exactly one tenth of the washed and dried pectin into a 250 ml Erlenmeyer flask and moisten with 2 ml 96% ethanol. Place the flask on a magnetic stirrer and slowly add 100 ml of boiled and cooled deionised water. Avoid splashing. Stir until all the pectin is completely dissolved. Add five drops of the solution of phenolphthalein and titrate with 0.1 N sodium hydroxide. The volume is recorded as V₁ in ml. Add 20.00 ml of 0.5 N sodium hydroxide, and shake vigorously. Allow the content to rest for 15 minutes in order to saponify the ester groups. Add 20.00 ml 0.5 N hydrochloric acid and shake until the pink color disappears. Add three drops of the solution of phenolphthalein and titrate with 0.1 N sodium hydroxide until achieving a faint persisting pink color, recording the volume of 0.1 N sodium hydroxide required as V₂ ml.

$$\% DE = \frac{{V}_{2} - {V}_{0}}{{V}_{1} + {V}_{2} - {V}_{0}} * 100.$$

### Determination of Intrinsic Viscosity

Intrinsic viscosity was determined by extrapolation the function reduced viscosity divided by the pectin concentration to zero concentration. At a given concentration the reduced viscosity was:

$$\frac{{η}_{c} - {η}_{r}}{{η}_{r}}$$

Where η_{c} is the viscosity of the pectin solution, ηᵣ is the viscosity of the solvent (1% aqueous solution of sodium polyphosphate, pH 4.75). The viscosities were measured on a Viscometer C (Haake, Gmbh), at 21.0° C., with a glass ball. The determined intrinsic viscosity was then used to calculate the molecular weight of pectins using the Mark-Houwink equation.

### Determination of CSP Ratio (CSPR)

2.00 g pectin was dissolved in approx. 90 g demineralised water at 70° C. After cooling to room temperature, pH was increased to 4.0 by addition of 20% by weight sodium carbonate solution during stirring. Demineralised water was added to 100.0 g total weight and the sample was mixed.

15.00 g of the sample was slowly mixed into 30.00 g 80% by weight aqueous 2-propanol in a 50 ml centrifuge screw cap tube to precipitate the pectin. After one hour with frequent shaking, the precipitated material was collected after centrifugation at 2800 g for 20 minutes and decantation of the centrifugate. To the precipitated material was added approx. 25 ml 60% by weight aqueous 2-propanol and it was thoroughly mixed. After one hour with frequent mixing, the precipitate was separated as above. The wash with 60% by weight 2-propanol was repeated once again and after final separation excess liquid was pressed off by hand and the pressed residue transferred to a tray and dried over night at 60° C. in a ventilated oven. The isolated pectin was weighed (a gram) and the dry matter a′ was determined in the material by drying at 105° C. for 2½ hours.

In a 50 ml centrifuge screw cap tube was added 25.00 g 60 mM CaCl₂) in demineralised water containing 16% by weight 2-propanol and mixed with 25.00 g of the prepared pectin sample. The mixture was shaken at lowest speed on an IKA MTS2 Schütler, which was connected to a timer to allow mixing for 1 out of 15 minutes. After 24 hours the mixture was centrifuged at 2800 g for 20 minutes and the liquid siphoned off through a cloth. The precipitated material in the centrifuge tube was weighed and an equal amount of 30 mM CaCl₂) in demineralised water containing 8% by weight 2-propanol was added and mixed. The tube was slowly shaken for another 24 hours and centrifuged at 2800 g for 20 minutes and the liquid siphoned off through a cloth. The wash of precipitated CSP was repeated once more. The residue in the tube was finally mixed with two parts of 80% by weight aqueous 2-propanol, and after one hour separated on the cotton cloth, and after draining transferred back to the tube and mixed again twice with equal amount of 60% by weight aqueous 2-propanol and after one hour drained and pressed in the cloth. The press residue was transferred to a tray and dried overnight at 60° C. in a ventilated oven. The residue was weighed (b gram) and the dry matter b′ of the material was determined by drying at 105° C. for 2½ hours.

CSP ratio was determined from:

$$CSP ratio = \frac{b * {b}^{′}}{a * {a}^{′}}$$

CSPR-figures above 1.0 can be achieved because the precipitation of particular small pectin molecules in presence of calcium is more quantitative than the 2-propanol precipitation. A CSPR figure above 1.0 indicates that the sample is pure calcium sensitive pectin.

### Determination of Activity of Polygalacturonase Enzymes

Several polygalacturonase-containing enzyme products were compared for the ability to improve pectin functionality. Initially their pectinase activities were determined in enzyme reactions containing pectin obtained from the process described in Example 1 below (without enzyme treatment).
a) Pectin is rehydrated in water to 0.5% (w/v) at room temperature.
b) The pH is adjusted by the addition of sodium acetate to 4.0.
c) The temperature is maintained at 40° C. during the enzymatic reactions.
d) Enzyme is added to time=0 and incubated with the pectin solution for different periods of time, before enzyme is inactivated at 95° C.
e) The quantity of reducing ends generated during the time of enzyme reaction is measured using para-hydroxybenzoic acid hydrazide (PAHBAH). By heat and alkaline conditions, the reducing end groups react with the colorless PAHBAH, whereby PAHBAH is oxidized. The complex is yellow and is quantified by spectrophotometry at 410 nm. Galacturonic acid is used for standard curve.
f) The activity is given as units/ml. One unit will release 1.0 micromole of reducing ends from pectin per minute at pH 4.0 at 40° C.

| Enzyme | Source | Units/ml |
|---|---|---|
| Rapidase Smart | DSM | 2395 |
| Rapidase Smart Plus | DSM | 2595 |
| Rohament PL | Rohm | 2030 |
| | GmbH | |
| Endo-PG M2 | Megazymes | 848 |

The invention is further described in the examples below.

### EXAMPLES

### Example 1: Combined Concentrated Pectin Treated with Polygalacturonase

### Materials

Citrus peel raw materials were extracted in a water:peel (w:p) ratio of 27 at 70° C. at pH 2.0 for three hours. Excess liquid was then separated by sieving and the first residue was washed for one hour at 70° C. with water added to constitute a w:p ratio of 12. After a second sieving the second residue including the sludge from centrifugation of the separated liquid was further extracted at 70° C. for two hours with 15 g oxalic acid monohydrate per kg citrus peel at pH 3.8±0.2 (adjusted with sodium carbonate solution) and w:p ratio of 19. The mixture was then separated by centrifugation. The first and second extracts were combined and separated by centrifugation.

The liquid phases from the centrifugations were mixed and clarified by passing the liquid through a vacuum filter with a layer of Clarcel DIT-R filter aid. The clarified juice was ion-exchanged through a weak-acidified Amberlite C200 resin, essentially in sodium form. The refined pectin juice was concentrated by ultrafiltration to 1.5-2.0% pectin concentration.

The pectin concentrate was treated with different amounts of polygalacturonase (nL enzyme/kg concentrate). All reactions were performed for 30 minutes at pH 3.5 and 50° C. Analysis of the treated pectins showed that increasing polygalacturonase dose leads to gradually decreasing pectin reactivity as expressed by CSPR.

TABLE 1
Characteristics of pectin samples 3826-109-X.

| Sample | Enzyme | % | MW | |
|---|---|---|---|---|
| Name | Dose nL/kg | DE | kDa | CSPR |
| 3826-109-1 | 100 | 67.8 | 90 | 0.645 |
| 3826-109-2 | 50 | 67.7 | 99 | 0.633 |
| 3826-109-3 | 38 | 67.6 | 104 | 0.705 |
| 3826-109-4 | 25 | 67.5 | 106 | 0.689 |
| 3826-109-5 | 15 | 67.6 | 120 | 0.808 |
| 3829-109-6 | 10 | 67.8 | 118 | 0.824 |
| 3826-109-8 | 0 | 67.8 | 122 | 0.853 |

### Rheological Characterization

Gels containing 0.65% by weight of pectin samples 1, 2, 4, 6 and 8 (Table 1), respectively, at pH 3.2 containing 65% soluble solids (sucrose, 320 mg K/kg jelly, 100 mg Ca/kg jelly were cooled from 95° C. to 20° C. at a rate of 2° C./minute. During cooling, the loss modulus and the storage modulus was measured using an Anton Paar Physica MCR301 rheometer equipped with CC27—SN19218 18487.

The data obtained from this experiment showed that the phase angle at 90° C. was reduced while the gelling temperature increased in gels containing pectin samples treated with decreasing amounts of polygalacturonase.

Conversely, the decreasing gelling temperature seen with increasing amounts of polygalacturonase is interpreted as reduced pectin reactivity brought about by reducing the galacturonic acid block structure in pectin using treatment with polygalacturonase. In selected applications, the results of the described polygalacturonase treatment provide an advantage both when the final pectin product is the resulting high ester pectin and when the high ester pectin is used as pre-cursor for de-esterification and/or amidation.

Table 2: Phase angle at 90° C. and crossover temperature for pectin treated with varying amounts of polygalacturonase.

TABLE 3
Results from rheology experiment.

| Sample Number | Phase angle at 90° C. | Crossover/° C. |
|---|---|---|
| 3826-109-1 | 62.1 | 77 |
| 3826-109-2 | 58.8 | 80 |
| 3826-109-4 | 53.1 | 80 |
| 3826-109-6 | 47.8 | 85 |
| 3826-109-8 | 46.4 | 86 |

### Example 2: Combined Treatment with Polygalacturonase and Pectin Methyl Esterase

### Materials

Citrus peel raw materials were extracted in a manner similar to that described in example 1 to generate two types of pectin precursor samples; the first sample (3834-73) collected after one extraction step with an aqueous solution of a pH 2.0; the second sample (3758-44) collected after two extraction steps as described in example 1 resulting in a combined pectin extract comprising more calcium-sensitive pectin than the 3834-73 sample (see table 4).

The liquid samples from extractions were clarified, ion-exchanged and concentrated essentially as described in example 1 but to 2.5-3.0% pectin concentration.

The pectin precursor concentrates were treated with polygalacturonase (PG)(3834-69) with an enzyme dosage of 0.4 U/L pectin concentrate, or a combination of pectin methyl esterase and polygalacturonase (PME/PG) with enzyme dosages of 268,9 U/L pectin concentrate (PME) and 0.05 U/L pectin concentrate (PG) to generate high ester (HE) pectin or low-ester (LC) pectin, respectively. Reactions were carried out at pH 3.8 and 50° C. and using pH-stat titration to reach different % DE in reactions containing PME. After reaction, pectin was precipitated by addition of 1.5 volumes IPA and washed in 60% IPA, dried and milled.

### Structural and Functional Analysis

Analysis of the treated pectin showed that a 2-step extraction (3758-44) provides a pectin with lower % DE and increased calcium sensitivity as compared to pectin from a 1-step acid extraction (3834-73). This can be reversed by treatment with polygalacturonase which provides the extra yield obtained by a 2-step extraction and a functionality of pectin that resembles that from 1-step extraction (3758-73) (table 4).

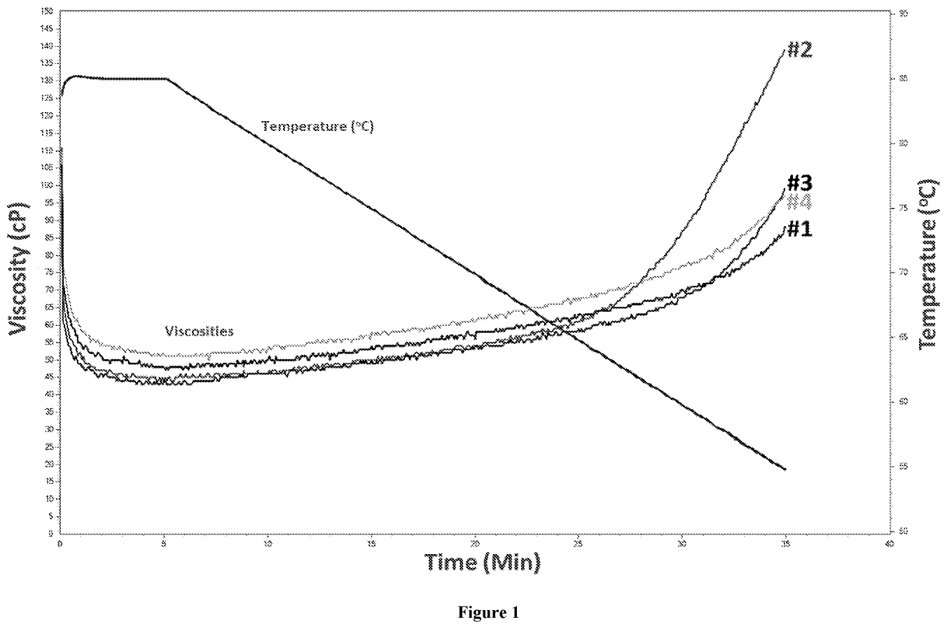

LC pectin products can be produced by a de-esterification after a 1-step acid pectin extraction using pectin methyl esterase (PME) to obtain the structural and functional parameters as given in table 4 (#1 4963430719). By combining the use of PME to de-esterify with PG to reduce the calcium sensitivity, it is possible to obtain LC pectin from a 2-step extraction procedure providing the same structural and functional parameters as a PME treatment of pectin from 1-step extraction (table 4 and FIG. 1, compare #1, #2, #3 and #4). Thus, the increased calcium sensitivity of pectin extracted by the 2-step extraction procedure can be reduced when producing LC pectin when using a combination of PME and PG treatment. Functional parameters were measured by gel strength analysis and by Rapid Viscosity Analyzer (RVA).

TABLE 4
Characteristics of pectin samples after PG or PME/PG treatment.

| | % | | Calcium | |
|---|---|---|---|---|
| Precursor pectin | DE | | sensitivity | |
| 3834-73 (1-step) | 71.0 | | 2.1 | |
| 3758-44 (2-step) | 65.1 | | 4.4 | |
| 3758-73 (2-step with PG) | 67.6 | | 2.5 | |
| | | RVA | RVA | Texture |
| | % | Viscosity | Viscosity | measurement |
| LC Pectin | DE | 85° C. | 55° C. | Gel strength |
| #1 4963430719 (1-step | 34.9 | 48 | 88 | 11.3 |
| with PME) | | | | |
| #2 3834-126-8 (2-step | 28.0 | 44 | 139 | 12.6 |
| with PME/PG) | | | | |
| #3 3834-126-7 (2-step | 33.2 | 44 | 99 | 9.5 |
| with PME/PG) | | | | |
| #4 3834-126-6 (2-step | 35.7 | 51 | 97 | 13.7 |
| with PME/PG) | | | | |

All publications mentioned in the above specification are herein incorporated by reference. Various modifications and variations of the described methods and composition of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in chemistry applied in food industry or related fields are intended to be within the scope of the following claims.

1. A process for reducing the calcium reactivity of pectin extracted from a pectin-containing raw material containing a high proportion of calcium-sensitive pectin, the process comprising the steps of:
(a) subjecting a pectin-containing raw material containing a high proportion of calcium-sensitive pectin to a first extraction procedure using a first aqueous solution of a pH in the range of 1.0-2.5, followed by separation of a first pectin-containing residue from the first aqueous solution, resulting in a first extract comprising both calcium-sensitive and calcium-tolerant pectin,
(b) subjecting the first pectin-containing residue to a second extraction procedure using a second aqueous solution of a pH in the range of 3.0-6.0, followed by separation of a second pectin-containing residue from the second aqueous solution, resulting in a second extract comprising calcium-sensitive pectin, and
(c) subjecting the second extract of step (b) or the combined first and second extracts of step (a) and (b) to treatment with an enzyme capable of depolymerizing pectin before or during purification of the extracts resulting in a pectin fraction comprising calcium-tolerant pectin with a lower molecular weight than the pectin present in the pectin-containing raw material.

2. The process of claim 1, wherein the pectin-containing raw material is obtained from citrus fruits, apples, sugar beets, sunflower heads, carrots, onions, peaches, grape berries, mangos, guavas, squashes, pumpkins, tomatoes, apricots, bananas or potatoes.

3. The process of claim 1, wherein the pectin-containing raw material is obtained from the peel of a citrus fruit.

4. The process of claim 1, wherein the enzyme is a polygalacturonase.

5. The process of claim 1, wherein the enzyme is an exo-polygalacturonase and/or an endo-polygalacturonase.

6. The process of claim 1, wherein the enzyme is a pectate lyase.

7. The process of claim 1, wherein the calcium-tolerant pectin prepared in step (c) has a molecular weight which is about 5-100 kDa lower than the molecular weight of the pectin of extract (b) or the combined first and second extracts of step (a) and (b).

8. The process of claim 1, wherein purification of the combined extracts comprises the steps of centrifugation, clarification, filtration, ion exchange, concentration and precipitation.

9. The process of claim 1, wherein step (c) is carried out before or after centrifugation of the second extract of step (b) or the combined first and second extracts of step (a) and (b).

10. The process of claim 1, wherein step (c) is carried out before clarification and filtration of the second extract of step (b) or the combined first and second extracts of step (a) and (b).

11. The process of claim 1, wherein step (c) is carried out after concentration of the second extract of step (b) or the combined first and second extracts of step (a) and (b).

12. The process of claim 1, wherein step (c) is carried out at a temperature in the range of about 40-80° C.

13. The process of claim 1, wherein step (c) is carried out at a pH in the range of 2.0-5.5.

14. The process of claim 1, wherein step (c) is carried out for 0.5-20 hours.

15. The process of claim 1, wherein the amount of the enzyme added to the second extract or combined first and second extracts in step (c) before or after centrifugation is 0.001-2 units/L extract.

16. The process of claim 1, wherein the amount of the enzyme added to the second extract or combined first or second extracts in step (c) after concentration is 0.005-10 units/L concentrate.

17. The process of claim 1, wherein step (c) further comprises subjecting the second extract or combined first and second extracts to treatment with one or more enzymes capable of de-esterifying pectin resulting in combined extracts comprising less calcium reactive pectin with both a reduced molecular weight and reduced degree of esterification compared to the pectin present in the pectin-containing raw material.

18. The process of claim 17, wherein the step (C) comprises subjecting the second extract or combined first and second extracts to treatment with a combination of a polygalacturonase and a pectin methyl esterase, a pectin acetyl esterase, a rhamnogalacturonase, a galactanase, an arabinase or a rhamnogalacturonan hydrolase.

19. The process of claim 17, wherein the de-esterifying enzyme is in a range of 2-10,000 units/L extract.

20. The process of claim 1, wherein:
the first pectin-containing reside is subjected to a washing step before being subjected to the second extraction procedure, and
the second aqueous solution comprises a chelating agent.
